(19) United States
(12) Patent Application Publication
(10) Pub. No.: US 2009/0005415 A1
(43) Pub. Date: Jan. 1, 2009

(54) 1,2-Diarylimidazoles for Use as Cb1 Modulators

(76) Inventors: Matti Ahlqvist, Molndal (SE); Leifeng Cheng, Molndal (SE); Robert Lundqvist, Molndal (SE); Henrik Sorensen, Molndal (SE)

Correspondence Address: MORGAN LEWIS & BOCKIUS LLP, 1111 PENNSYLVANIA AVENUE NW, WASHINGTON, DC, 20004, US

(21) Appl. No.: 12/066,946

(22) PCT Filed: Sep. 12, 2006
(86) PCT No.: PCT/GB2006/003356

§ 371 Date: Aug. 4, 2008

(30) Foreign Application Priority Data
Sep. 15, 2005 (GB) 0518817.2

(51) Int. Cl. A61K 31/454; C07D 401/02; A61K 31/4545; A61P 25/00; C07D 401/14

(52) U.S. Cl. 514/318; 546/210; 546/194; 514/326

(57) ABSTRACT

Salts of 1,2-diarylimidazole-4-carboxamides and processes for preparing such compounds, their use in the treatment of obesity, psychiatric and neurological disorders, to methods for their therapeutic use and to pharmaceutical compositions containing them.

### FIELD OF THE INVENTION

The present invention relates to certain salts of 1,2-diarylimidazole-4-carboxamides, to processes for preparing such compounds, to their use in the treatment of obesity, psychiatric and neurological disorders, to methods for their therapeutic use and to pharmaceutical compositions containing them.

### BACKGROUND OF THE INVENTION

It is known that certain CB, modulators (known as antagonists or inverse agonists) are useful in the treatment of obesity, psychiatric and neurological disorders (WO01170700 and EP 656354).

WO04/60367 and WO2004/099130 disclose that certain diaryl imidazoles and triazoles are useful as COX-1 inhibitors useful in the treatment of inflammation. Compounds exemplified in these applications are disclaimed from the claims of the present invention.

DD 140966 discloses that certain imidazolecarboxylic acid anilides are useful as plant growth regulators. Compounds exemplified in this application are disclaimed from the claims of the present invention.

WO 03/007887 and WO03/075660 disclose certain 4,5-diarylimidazole-2-carboxamides as CB, modulators.

WO03/27076 and WO 03/63781 disclose certain 1,2-diarylimidazole-4-carboxamides which are CB, modulators. Compounds exemplified in these applications are disclaimed from the claims of the present invention.

WO03/40107 discloses certain 1,2-diarylimidazole-4-carboxamides as being useful in the treatment of obesity and obesity-related disorders.

Co-pending PCT application No. PCT/GB2005/001153 discloses compounds of formula A

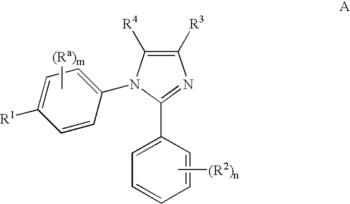

and pharmaceutically acceptable salts thereof, in which

R¹ represents a) a C₁₋₁₀alkoxy group optionally substituted by one or more fluoro b) a group of formula phenyl(CH₂)ₚO— in which p is 1, 2 or 3 and the phenyl ring is optionally substituted by 1, 2 or 3 groups represented by Z, c) a group R⁵S(O)₂O or R⁵S(O)₂NH in which R⁵ represents a C₁₋₁₀alkyl group optionally substituted by one or more fluoro, or R⁵ represents phenyl or a heteroaryl group each of which is optionally substituted by 1, 2 or 3 groups represented by Z or d) a group of formula (R⁶)₃ Si in which R⁶ represents a C₁₋₆alkyl group which may be the same or different;

R^{a} represents halo, a C₁₋₃alkyl group or a C₁₋₃alkoxy group;
m is 0, 1, 2 or 3;
R² represents a C₁₋₃alkyl group, a C₁₋₃alkoxy group, hydroxy, nitro, cyano or halo is n is 0, 1, 2 or 3;
R³ represents
a) a group X—Y—NR⁷R⁸
in which X is CO or SO₂,
Y is absent or represents NH optionally substituted by a C₁₋₃alkyl group;
and R⁷ and R⁸ independently represent:
a C₁₋₆alkyl group optionally substituted by 1, 2, or 3 groups represented by W;
a C₃₋₁₅cycloalkyl group optionally substituted by 1, 2, or 3 groups represented by W;
an optionally substituted (C₃₋₁₅cycloalkyl)C₁₋₃alkylene group optionally substituted by 1, 2, or 3 groups represented by W;
a group —(CH₂)ᵣ(phenyl)ₛ in which r is 0, 1, 2, 3 or 4, s is 1 when r is 0 otherwise s is 1 or 2 and the phenyl groups are optionally independently substituted by one, two or three groups represented by Z;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;
a group —(CH₂)ₜHet in which t is 0, 1, 2, 3 or 4, and the alkylene chain is optionally substituted by one or more C₁₋₃alkyl groups and Het represents a heteroaryl group optionally substituted by one, two or three groups selected from a C₁₋₅alkyl group, a C₁₋₅alkoxy group or halo wherein the alkyl and alkoxy group are optionally independently substituted by one of more fluoro;
or R⁷ represents H and R⁸ is as defined above;
or R⁷ and R⁸ together with the nitrogen atom to which they are attached represent a saturated or partially unsaturated 5 to 8 membered heterocyclic group containing one nitrogen and optionally one of the following: oxygen, sulphur or an additional nitrogen;
wherein the heterocyclic group is optionally substituted by one or more C₁₋₃alkyl groups, hydroxy, fluoro or benzyl;
or b) oxazolyl, isoxazolyl, thiazolyl, isothiazolyl, oxadiazolyl, thiadiazolyl, pyrrolyl, pyrazolyl, imidazolyl, triazolyl, tetrazolyl, thienyl, furyl or oxazolinyl, each optionally substituted by 1, 2 or 3 groups Z;
R⁴ represents H, a C₁₋₆alkyl group, a C₁₋₆alkoxy group or a C₁₋₆alkoxyC₁₋₆alkylene group which contains a maximum of 6 carbon atoms, each of which groups is optionally substituted by one or more fluoro or cyano;
Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, C₁₋₃alkylsulphonyl, C₁₋₃alkoxycarbonyl, carboxy, cyano, carbamoyl, mono or di C₁₋₃alkyl carbamoyl and acetyl; and
W represents hydroxy, fluoro, a C₁₋₃alkyl group, a C₁₋₃alkoxy group, amino, mono or di C₁₋₃alkylamino, or a heterocyclic amine selected from morpholinyl, pyrrolidinyl, piperidinyl or piperazinyl in which the heterocyclic amine is optionally substituted by a C₁₋₃alkyl group or hydroxyl;
with the proviso that when n is 1 then R² is not methoxy in either the 2-position or the 4-position of the phenyl ring and the further proviso that R¹ is not methylsulfonylamino, methoxy or CF₃O—.

Salts are mentioned in general terms in PCT/GB2005/001153 in the following way: “Pharmaceutically acceptable salt”, where such salts are possible, includes both pharmaceutically acceptable acid and base addition salts. A suitable pharmaceutically acceptable salt of a compound of Formula A is, for example, an acid-addition salt of a is compound of Formula A which is sufficiently basic, for example an acid-addition salt with an inorganic or organic acid such as hydrochloric, hydrobromic, sulphuric, trifluoroacetic, citric or maleic acid; or, for example a salt of a compound of Formula A which is sufficiently acidic, for example an alkali or alkaline earth metal salt such as a sodium, calcium or magnesium salt, or an ammonium salt, or a salt with an organic base such as methylamine, dimethylamine, trimethylamine, piperidine, morpholine or tris-(2-hydroxyethyl)amine.

Further salts with suitable properties for pharmaceutical formulation have now been found.

In the formulation of drug compositions, it is important for the drug substance to be in a form in which it can be conveniently handled and processed. This is of importance, not only from the point of view of obtaining a commercially viable manufacturing process, but also from the point of view of subsequent manufacture of pharmaceutical formulations comprising the active compound.

Further, in the manufacture of drug compositions, it is important that a reliable, reproducible and constant plasma concentration profile of drug is provided following administration to a patient.

Chemical stability, solid-state stability, and “shelf life” of the active ingredients are also very important factors. The drug substance, and compositions containing it, should preferably be capable of being effectively stored over appreciable periods of time, without exhibiting a significant change in the active component's physico-chemical characteristics (e.g. its chemical composition, density, hygroscopicity and solubility).

Moreover, it is also important to be able to provide the drug in a form that is as chemically pure as possible.

The skilled person will appreciate that, typically, if a drug can be readily obtained in a stable form, such as a stable crystalline form, advantages may be provided, in terms of ease of handling, ease of preparation of suitable pharmaceutical formulations, and a more reliable solubility profile.

### DESCRIPTION OF THE INVENTION

The present invention provides a compound selected from:
1-(4-benzyloxyphenyl)-2-(2,4-dichlorophenyl)-5-methyl-1H-imidazole-4-carboxylic acid piperidin-1-ylamide;
ethanesulfonic acid 4-[2-(2,4-dichlorophenyl)-5-methyl-4-(piperidin-1-ylcarbamoyl)-imidazol-1-yl]phenyl ester;
propane-1-sulfonic acid 4-[2-(2,4-dichlorophenyl)-5-methyl-4-(piperidin-1-ylcarbamoyl)-imidazol-1-yl]phenyl ester;
butane-1-sulfonic acid 4-[2-(2,4-dichlorophenyl)-5-methyl-4-(piperidin-1-ylcarbamoyl)-imidazol-1-yl]phenyl ester;
2-(2,4-dichlorophenyl)-5-methyl-1-[4-(4,4,4-trifluorobutoxy)phenyl]-1H-imidazole-4-carboxylic acid piperidin-1-ylamide;
3,3,3-trifluoropropane-1-sulfonic acid 4-[2-(2,4-dichlorophenyl)-5-methyl-4-(piperidin-1-ylcarbamoyl)imidazol-1-yl]phenyl ester;
4,4,4-trifluorobutane-1-sulfonic acid 4-[2-(2,4-dichlorophenyl)-5-methyl-4-(piperidin-1-ylcarbamoyl)imidazol-1-yl]phenyl ester;
4-{2-(2,4-dichlorophenyl)-5-methyl-4-[(piperidin-1-ylamino)carbonyl]-1H-imidazol-1-yl}phenyl thiophene-2-sulfonate;
4-{2-(2,4-dichlorophenyl)-5-methyl-4-[(piperidin-1-ylamino)carbonyl]-1H-imidazol-1-yl}phenyl pyridine-3-sulfonate;
4-{2-(2,4-dichlorophenyl)-5-methyl-4-[(piperidin-1-ylamino)carbonyl]-1H-imidazol-1-yl}phenyl pyridine-3-sulfonate;
4-{2-(2,4-dichlorophenyl)-5-methyl-4-[(piperidin-1-ylamino)carbonyl]-1H-imidazol-1-yl}phenyl 3-methylbutane-1-sulfonate;
4-{2-(2,4-dichlorophenyl)-5-methyl-4-[(piperidin-1-ylamino)carbonyl]-1H-imidazol-1-yl}phenyl 3,3-dimethylbutane-1-sulfonate;
4-[2-(2,4-dichlorophenyl)-5-methyl-4-({[5-(trifluoromethyl)pyridin-2-yl]amino}carbonyl)-1H-imidazol-1-yl]phenyl 3,3,3-trifluoropropane-1-sulfonate; and
4-[2-(2,4-dichlorophenyl)-5-methyl-4-({[5-(trifluoromethyl)pyridin-2-yl]amino}carbonyl)-1H-imidazol-1-yl]phenyl 3-methylbutane-1-sulfonate;
   in the form of a methanesulphonate salt (mesylate salt), a hemi-1,5-naphthalenedisulphonate salt, a hemi-1,2-ethanedisulfonic acid salt, a 1,2-ethanedisulfonic acid salt, a hydrochloride salt, an ethylsulphonate salt, a nitrate salt, a sulphate salt or a hydrogen sulphate salt.

In a particular aspect the present invention provides one or more of the following:
1-(4-benzyloxyphenyl)-2-(2,4-dichlorophenyl)-5-methyl-1H-imidazole-4-carboxylic acid piperidin-1-ylamide hydrochloride;
1-(4-benzyloxyphenyl)-2-(2,4-dichlorophenyl)-5-methyl-1H-imidazole-4-carboxylic acid piperidin-1-ylamide mesylate;
1-(4-benzyloxyphenyl)-2-(2,4-dichlorophenyl)-5-methyl-1H-imidazole-4-carboxylic acid piperidin-1-ylamide hemi-1,5-naphthalenedisulphonate;
ethanesulfonic acid 4-[2-(2,4-dichlorophenyl)-5-methyl-4-(piperidin-1-ylcarbamoyl)-imidazol-1-yl]phenyl ester hydrochloride;
ethanesulfonic acid 4-[2-(2,4-dichlorophenyl)-5-methyl-4-(piperidin-1-ylcarbamoyl)-imidazol-1-yl]phenyl ester mesylate;
ethanesulfonic acid 4-[2-(2,4-dichlorophenyl)-5-methyl-4-(piperidin-1-ylcarbamoyl)-imidazol-1-yl]phenyl ester hemi-1,5-naphthalenedisulphonate;
4,4,4-trifluorobutane-1-sulfonic acid 4-[2-(2,4-dichlorophenyl)-5-methyl-4-(piperidin-1-ylcarbamoyl)imidazol-1-yl]phenyl ester hydrochloride;
4,4,4-trifluorobutane-1-sulfonic acid 4-[2-(2,4-dichlorophenyl)-5-methyl-4-(piperidin-1-ylcarbamoyl)imidazol-1-yl]phenyl ester-4,4,4-trifluorobutane-1-sulfonic acid mesylate;
4-[2-(2,4-dichlorophenyl)-5-methyl-4-(piperidin-1-ylcarbamoyl)imidazol-1-yl]phenyl ester hemi-1,5-naphthalenedisulphonate;
4,4,4-trifluorobutane-1-sulfonic acid 4-[2-(2,4-dichlorophenyl)-5-methyl-4-(piperidin-1-ylcarbamoyl)imidazol-1-yl]phenyl ester hydrogen sulphate;
pyridine-3-sulfonic acid 4-[2-(2,4-dichlorophenyl)-5-methyl-4-(piperidin-1-ylcarbamoyl)-imidazol-1-yl]phenyl ester hydrochloride;
pyridine-3-sulfonic acid 4-[2-(2,4-dichlorophenyl)-5-methyl-4-(piperidin-1-ylcarbamoyl)-imidazol-1-yl]phenyl ester hemi-1,5-naphthalenedisulphonate;
5-chlorothiophene-2-sulfonic acid 4-[2-(2,4-dichlorophenyl)-5-methyl-4-(piperidin-1-ylcarbamoyl)imidazol-1-yl]phenyl ester hydrochloride;
5-chlorothiophene-2-sulfonic acid 4-[2-(2,4-dichlorophenyl)-5-methyl-4-(piperidin-1-ylcarbamoyl)imidazol-1-yl]phenyl ester mesylate;
5-chlorothiophene-2-sulfonic acid 4-[2-(2,4-dichlorophenyl)-5-methyl-4-(piperidin-1-ylcarbamoyl)imidazol-1-yl]phenyl ester hemi-1,5-naphthalenedisulphonate;
5-chlorothiophene-2-sulfonic acid 4-[2-(2,4-dichlorophenyl)-5-methyl-4-(piperidin-1-ylcarbamoyl)imidazol-1-yl]phenyl ester hydrogen sulphate;
3,3-dimethylbutane-1-sulfonic acid 4-[2-(2,4-dichlorophenyl)-5-methyl-4-(piperidin-1-ylcarbamoyl)imidazol-1-yl]phenyl ester hydrochloride;
3,3-dimethylbutane-1-sulfonic acid 4-[2-(2,4-dichlorophenyl)-5-methyl-4-(piperidin-1-ylcarbamoyl)imidazol-1-yl]phenyl ester mesylates;
3,3-dimethylbutane-1-sulfonic acid 4-[2-(2,4-dichlorophenyl)-5-methyl-4-(piperidin-1-ylcarbamoyl)imidazol-1-yl]phenyl ester hemi-1,5-naphthalenedisulphonate;
thiophene-2-sulfonic acid 4-[2-(2,4-dichlorophenyl)-5-methyl-4-(piperidin-1-ylcarbamoyl)imidazol-1-yl]phenyl ester hydrochloride;
thiophene-2-sulfonic acid 4-[2-(2,4-dichlorophenyl)-5-methyl-4-(piperidin-1-ylcarbamoyl)imidazol-1-yl]phenyl ester hemi-1,5-naphthalenedisulphonate;
thiophene-2-sulfonic acid 4-[2-(2,4-dichlorophenyl)-5-methyl-4-(piperidin-1-ylcarbamoyl)imidazol-1-yl]phenyl ester hydrogen sulphate;
3,3,3-trifluoropropane-1-sulfonic acid 4-[2-(2,4-dichlorophenyl)-5-methyl-4-(piperidin-1-ylcarbamoyl)imidazol-1-yl]phenyl ester hydrogen sulphate;
3,3,3-trifluoropropane-1-sulfonic acid 4-[2-(2,4-dichlorophenyl)-5-methyl-4-(piperidin-1-ylcarbamoyl)imidazol-1-yl]phenyl ester mesylate;
3,3,3-trifluoropropane-1-sulfonic acid 4-[2-(2,4-dichlorophenyl)-5-methyl-4-(piperidin-1-ylcarbamoyl)imidazol-1-yl]phenyl ester hemi-1,5-naphthalenedisulphonate;
3,3,3-trifluoropropane-1-sulfonic acid 4-[2-(2,4-dichlorophenyl)-5-methyl-4-(piperidin-1-ylcarbamoyl)imidazol-1-yl]phenyl ester ethylsulphonate;
propane-1-sulfonic acid 4-[2-(2,4-dichlorophenyl)-5-methyl-4-(piperidin-1-ylcarbamoyl)imidazol-1-yl]phenyl ester hydrochloride;
propane-1-sulfonic acid 4-[2-(2,4-dichlorophenyl)-5-methyl-4-(piperidin-1-ylcarbamoyl)imidazol-1-yl]phenyl ester mesylates; or
propane-1-sulfonic acid 4-[2-(2,4-dichlorophenyl)-5-methyl-4-(piperidin-1-ylcarbamoyl)imidazol-1-yl]phenyl ester hemi-1,5-naphthalenedisulphonate.

It will be understood that the present invention includes one or any combination of more than one of the above salts.

We have found that certain compounds of the invention have the advantage that they may be prepared in crystalline form.

According to a further aspect of the invention there is provided a compound of the invention in substantially crystalline form.

Although we have found that it is possible to produce compounds of the invention in forms which are greater than 80% crystalline, by “substantially crystalline” we include greater than 20%, preferably greater than 30%, and more preferably greater than 40% (e.g. greater than any of 50, 60, 70, 80 or 90%) crystalline.

According to a further aspect of the invention there is also provided a compound of the invention in partially crystalline form. By “partially crystalline” we include 5% or between 5% and 20% crystalline.

The degree (%) of crystallinity may be determined by the skilled person using X-ray powder diffraction (XRPD). Other techniques, such as solid state NMR, FT-IR, Raman spectroscopy, differential scanning calorimetry (DSC) and microcalorimetry, may also be used.

Compounds of the invention, and particularly crystalline compounds of the invention, may have improved stability when compared to compounds disclosed in PCT/GB2005/001153.

The term “stability” as defined herein includes chemical stability and solid state stability.

By “chemical stability”, we include that it may be possible to store compounds of the invention in an isolated form, or in the form of a formulation in which it is provided in admixture with pharmaceutically acceptable carriers, diluents or adjuvants (e.g. in an oral dosage form, such as a tablet, capsule etc.), under normal storage conditions, with an insignificant degree of chemical degradation or decomposition.

By “solid state stability”, we include that it may be possible to store compounds of the invention in an isolated solid form, or in the form of a solid formulation in which it is provided in admixture with pharmaceutically acceptable carriers, diluents or adjuvants (e.g. in an oral dosage form, such as a tablet, capsule etc.), under normal storage conditions, with an insignificant degree of solid state transformation (e.g. crystallisation, recrystallisation, solid state phase transition, hydration, dehydration, solvatisation or desolvatisation).

Examples of “normal storage conditions” include temperatures of between minus 80 and plus 50° C. (preferably between 0 and 40° C. and more preferably room temperatures, is such as 15 to 30° C.), pressures of between 0.1 and 2 bars (preferably at atmospheric pressure), relative humidities of between 5 and 95% (preferably 10 to 60%), and/or exposure to 460 lux of UV/visible light, for prolonged periods (i.e. greater than or equal to six months). Under such conditions, compounds of the invention may be found to be less than 15%, more preferably less than 10%, and especially less than 5%, chemically degraded/decomposed, or solid state transformed, as appropriate. The skilled person will appreciate that the above-mentioned upper and lower limits for temperature, pressure and relative humidity represent extremes of normal storage conditions, and that certain combinations of these extremes will not be experienced during normal storage (e.g. a temperature of 50° C. and a pressure of 0.1 bar).

It may be possible to crystallise salts of compounds of the present invention with or without the presence of a solvent system (e.g. crystallisation may be from a melt, under supercritical conditions, or achieved by sublimation). However, it is preferable that crystallisation occurs from an appropriate solvent system.

According to a further aspect of the invention, there is provided a process for the preparation of a crystalline compound of the invention which comprises crystallising a compound of the invention from an appropriate solvent system.

Crystallisation temperatures and crystallisation times depend upon the salt that is to be crystallised, the concentration of that salt in solution, and the solvent system that is used.

Crystallisation may also be initiated and/or effected by way of standard techniques, for example with or without seeding with crystals of the appropriate crystalline compound of the invention.

Different crystalline forms of the compounds of the invention may be readily characterised using X-ray powder diffraction (XRPD) methods, for example as described hereinafter.

In order to ensure that a particular crystalline form is prepared in the absence of other crystalline forms, crystallisations are preferably carried out by seeding with nuclei and/or seed crystals of the desired crystalline form in substantially complete absence of nuclei and/or seed crystals of other crystalline forms. Seed crystals of appropriate compound may be prepared, for example, by way of slow evaporation of solvent from a is portion of solution of appropriate salt.

Compounds of the invention may be isolated using techniques which are well known to those skilled in the art, for example decanting, filtering or centrifuging.

Compounds may be dried using standard techniques.

Further purification of compounds of the invention may be effected using techniques, which are well known to those skilled in the art. For example impurities may be removed by way of recrystallisation from an appropriate solvent system. Suitable temperatures and times for the recrystallisation depend upon the concentration of the salt in solution, and upon the solvent system that is used.

When compounds of the invention are crystallised, or recrystallised, as described herein, the resultant salt may be in a form which has improved chemical and/or solid state stability, as mentioned hereinbefore.

Compounds of the invention have the advantage that they may be more efficacious, be less toxic, be longer acting, have a broader range of activity, be more potent, produce fewer side effects, be more easily absorbed, and/or have a better pharmacokinetic profile (e.g. higher oral bioavailability and/or lower clearance), than, and/or have other useful pharmacological, physical, or chemical, properties over, compounds known in the prior art.

Compounds of the invention may have the further advantage that they may be administered less frequently than compounds known in the prior art.

Compounds of the invention may also have the advantage that they are in a form which provides for improved ease of handling. Further, compounds of the invention have the advantage that they may be produced in forms which may have improved chemical and/or solid state stability (including e.g. due to lower hygroscopicity). Thus, such compounds of the invention may be stable when stored over prolonged periods.

Compounds of the invention may also have the advantage that they may be crystallised in good yields, in a high purity, rapidly, conveniently, and at a low cost.

It will also be understood that the compounds of the present invention may exist in solvated, for example hydrated, as well as unsolvated forms. It is to be understood that the present invention encompasses all such solvated and unsolvated forms.

### Methods of Preparation

The compounds of the invention may be prepared as outlined below. However, the invention is not limited to these methods.

The salts may be prepared by reacting a compound prepared as described in PCT/GB2005/001153 and in the examples section of this application with the appropriate acid, for example methanesulphonic acid, naphthalene-1,5-disulphonic acid, 1,2-ethanedisulfonic acid, sulphuric acid or hydrochloric acid in an inert solvent, for example butanone at a temperature in the range of 0-100° C. and isolating the solid salt. The salt may be isolated by cooling the reaction solution and optionally seeding the solution with the desired product and/or concentrating the solution. Optionally the product may be isolated by adding an antisolvent to a solution of the product in an inert solvent. The solid may be collected by methods known to those skilled in the art for example filtration or centrifugation.

Particularly a molar equivalent of acid with respect to the free base is used for methanesulphonic acid, ethanesulphonic acid, sulphuric acid and hydrochloric acid whereas for, 1,5-naphthalenedisulphonic, sulphuric acid and 1,2-ethanedisulfonic acid a half a molar equivalent is used with respect to the free base. It will be appreciated that a slight excess of either the basic compound or the acid may be employed. For example with sulphuric acid a ratio of 1 molar equivalent of acid to 0.5 molar equivalents of base may be employed.

The expression “inert solvent” refers to a liquid that dissolves or partially dissolves the free base and/or the acid and/or the product salt but does not react with the starting materials, reagents, intermediates or products in a manner that adversely affects the yield of the desired product.

In another aspect the present invention provides the compound obtainable by reacting one of the following:
1-(4-benzyloxyphenyl)-2-(2,4-dichlorophenyl)-5-methyl-1H-imidazole-4-carboxylic acid piperidin-1-ylamide;
ethanesulfonic acid 4-[2-(2,4-dichlorophenyl)-5-methyl-4-(piperidin-1-ylcarbamoyl)-imidazol-1-yl]phenyl ester;
propane-1-sulfonic acid 4-[2-(2,4-dichlorophenyl)-5-methyl-4-(piperidin-1-ylcarbamoyl)-imidazol-1-yl]phenyl ester;
butane-1-sulfonic acid 4-[2-(2,4-dichlorophenyl)-5-methyl-4-(piperidin-1-ylcarbamoyl)-imidazol-1-yl]phenyl ester;
2-(2,4-dichlorophenyl)-5-methyl-1-[4-(4,4,4-trifluorobutoxy)phenyl]-1H-imidazole-4-carboxylic acid piperidin-1-ylamide;
3,3,3-trifluoropropane-1-sulfonic acid 4-[2-(2,4-dichlorophenyl)-5-methyl-4-(piperidin-1-ylcarbamoyl)imidazol-1-yl]phenyl ester;
4,4,4-trifluorobutane-1-sulfonic acid 4-[2-(2,4-dichlorophenyl)-5-methyl-4-(piperidin-1-ylcarbamoyl)imidazol-1-yl]phenyl ester;
4-{2-(2,4-dichlorophenyl)-5-methyl-4-[(piperidin-1-ylamino)carbonyl]-1H-imidazol-1-yl}phenyl thiophene-2-sulfonate;
4-{2-(2,4-dichlorophenyl)-5-methyl-4-[(piperidin-1-ylamino)carbonyl]-1H-imidazol-1-yl}phenyl pyridine-3-sulfonate;
4-{2-(2,4-dichlorophenyl)-5-methyl-4-[(piperidin-1-ylamino)carbonyl]-1H-imidazol-1-yl}phenyl pyridine-3-sulfonate;
4-{2-(2,4-dichlorophenyl)-5-methyl-4-[(piperidin-1-ylamino)carbonyl]-1H-imidazol-1-yl}phenyl 3-methylbutane-1-sulfonate;
4-{2-(2,4-dichlorophenyl)-5-methyl-4-[(piperidin-1-ylamino)carbonyl]-1H-imidazol-1-yl}phenyl 3,3-dimethylbutane-1-sulfonate;
4-[2-(2,4-dichlorophenyl)-5-methyl-4-({[5-(trifluoromethyl)pyridin-2-yl]amino}carbonyl)-1H-imidazol-1-yl]phenyl 3,3,3-trifluoropropane-1-sulfonate; and
4-[2-(2,4-dichlorophenyl)-5-methyl-4-({[5-(trifluoromethyl)pyridin-2-yl]amino}carbonyl)-1H-imidazol-1-yl]phenyl 3-methylbutane-1-sulfonate;
   with either
   a) a molar equivalent hydrochloric acid or
   b) a molar equivalent methanesulphonic acid or
   c) a molar equivalent of sulphuric acid or
   d) a half molar equivalent of sulphuric acid or
   e) a half a molar equivalent of 1,5-naphthalenedisulphonic acid or
   f) a half a molar equivalent of 1,2-ethanedisulfonic acid or
   g) a molar equivalent of 1,2-ethanedisulfonic acid or
   h) a molar equivalent ethanesulphonic acid
   in butanone optionally in the presence of methanol and/or heptane.
   It will be understood by those skilled in the art that the molar equivalents used are within the range of experimental error and may include a slight excess of one of the reactants for example plus or minus 10% of the theoretical equivalent weight.
   In another aspect the present invention provides a compound of formula I

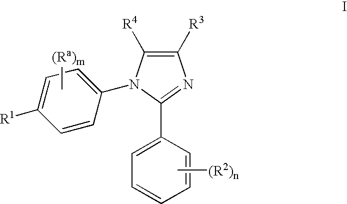

in which
R¹ represents a) a C₁₋₁₀alkoxy group optionally substituted by one or more fluoro b) a group of formula phenyl(CH₂)ₚO— in which p is 1, 2 or 3 and the phenyl ring is optionally substituted by 1, 2 or 3 groups represented by Z, c) a group R⁵S(O)₂O or R⁵S(O)₂NH in which R⁵ represents a C₁₋₁₀alkyl group optionally substituted by one or more fluoro, or R⁵ represents phenyl or a heteroaryl group each of which is optionally substituted by 1, 2 or 3 groups represented by Z or d) a group of formula (R⁶)₃ Si in which R⁶ represents a C₁₋₆alkyl group which may be the same or different;
R^{a} represents halo, a C₁₋₃alkyl group or a C₁₋₃alkoxy group;
m is 0, 1, 2 or 3;
R² represents a C₁₋₃alkyl group, a C₁₋₃alkoxy group, hydroxy, nitro, cyano or halo
n is 0, 1, 2 or 3;
R³ represents
a) a group X—Y—NR⁷R⁸
in which X is CO or SO₂,
Y is absent or represents NH optionally substituted by a C₁₋₃alkyl group;
and R⁷ and R⁸ independently represent:
a C₁₋₆alkyl group optionally substituted by 1, 2, or 3 groups represented by W;
a C₃₋₁₅cycloalkyl group optionally substituted by 1, 2, or 3 groups represented by W;
an optionally substituted (C₃₋₁₅cycloalkyl)C₁₋₃alkylene group optionally substituted by 1, 2, or 3 groups represented by W;
a group —(CH₂)ᵣ(phenyl), in which r is 0, 1, 2, 3 or 4, s is 1 when r is 0 otherwise s is 1 or 2 and the phenyl groups are optionally independently substituted by one, two or three groups represented by Z;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;
a group —(CH₂)ₜHet in which t is 0, 1, 2, 3 or 4, and the alkylene chain is optionally substituted by one or more C₁₋₃alkyl groups and Het represents a heteroaryl group optionally substituted by one, two or three groups selected from a C₁₋₅alkyl group, a C₁₋₅alkoxy group or halo wherein the alkyl and alkoxy group are optionally independently substituted by one of more fluoro;
or R⁷ represents H and R⁸ is as defined above;
or R⁷ and R⁸ together with the nitrogen atom to which they are attached represent a saturated or partially unsaturated 5 to 8 membered heterocyclic group containing one nitrogen and optionally one of the following: oxygen, sulphur or an additional nitrogen;
wherein the heterocyclic group is optionally substituted by one or more C₁₋₃alkyl groups, hydroxy, fluoro or benzyl;
or b) oxazolyl, isoxazolyl, thiazolyl, isothiazolyl, oxadiazolyl, thiadiazolyl, pyrrolyl, pyrazolyl, imidazolyl, triazolyl, tetrazolyl, thienyl, furyl or oxazolinyl, each optionally substituted by 1, 2 or 3 groups Z;
R⁴ represents H, a C₁₋₆alkyl group, a C₁₋₆alkoxy group or a C₁₋₆alkoxyC₁₋₆alkylene group which contains a maximum of 6 carbon atoms, each of which groups is optionally substituted by one or more fluoro or cyano;
Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, C₁₋₃alkylsulphonyl, C₁₋₃alkoxycarbonyl, carboxy, cyano, carbamoyl, mono or di C₁₋₃alkyl carbamoyl and acetyl; and
W represents hydroxy, fluoro, a C₁₋₃alkyl group, a C₁₋₃alkoxy group, amino, mono or di C₁₋₃alkylamino, or a heterocyclic amine selected from morpholinyl, pyrrolidinyl, piperidinyl or piperazinyl in which the heterocyclic amine is optionally substituted by a C₁₋₃alkyl group or hydroxyl;
with the proviso that when n is 1 then R² is not methoxy in either the 2-position or the 4-position of the phenyl ring and the further proviso that R¹ is not methylsulfonylamino, methoxy or CF₃O—;
in the form of a methanesulphonate salt (mesylate salt), a hemi-1,5-naphthalenedisulphonate salt, a hemi-1,2-ethanedisulfonic acid salt, a 1,2-ethanedisulfonic acid salt, an ethylsulphonate salt, or a nitrate salt.

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight.

Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

### Pharmacological Properties

The compounds of the invention are useful for the treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

The compounds are also potentially useful for the prevention or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

The compounds are also potentially useful for the prevention or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

The compounds are also potentially useful for the treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

The compounds are also potentially useful as agents in treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders. The compounds are also potentially useful as agents in treatment of (esophageal) achalasia.

In another aspect the present invention provides a compound of the invention as previously defined for use as a medicament.

In a further aspect the present invention provides the use of a compound of the invention in the preparation of a medicament for the treatment or prophylaxis of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a further aspect the present invention provides the use of a compound of the invention in the preparation of a medicament for the treatment or prophylaxis of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a further aspect the present invention provides the use of a compound of the invention in the preparation of a medicament for the treatment or prophylaxis of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

In a further aspect the present invention provides the use of a compound of the invention in the preparation of a medicament for the treatment or prophylaxis of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a further aspect the present invention provides the use of a compound of the invention in the preparation of a medicament for the treatment or prophylaxis of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

In a still further aspect the present invention provides a method comprising administering a pharmacologically effective amount of a compound of the invention to a patient in need thereof for the prophylaxis or treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a still further aspect the present invention provides a method comprising administering a pharmacologically effective amount of a compound of the invention to a patient in need thereof for the prophylaxis or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a still further aspect the present invention provides a method comprising administering a pharmacologically effective amount of a compound of the invention to a patient in need thereof for the prophylaxis or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

In a still further aspect the present invention provides a method comprising administering a pharmacologically effective amount of a compound of the invention to a patient in need thereof for the prophylaxis or treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a still further aspect the present invention provides a method comprising administering a pharmacologically effective amount of a compound of the invention to a patient in need thereof for the prophylaxis or treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

The compounds of the present invention are particularly suitable for the treatment of obesity or being overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items). The compounds of the invention are useful for the treatment of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, and neurological disorders such as dementia, neurological disorders (e.g. Multiple Sclerosis), Raynaud's syndrome, Parkinson's disease, Huntington's chorea and Alzheimer's disease. The compounds are also potentially useful for the treatment of immune, cardiovascular, reproductive and endocrine disorders, septic shock and diseases related to the respiratory and gastrointestinal systems (e.g. diarrhea). The compounds are also potentially useful as agents in treatment of extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms. The compounds may also eliminate the increase in weight that normally accompanies the cessation of smoking.

In another aspect the present invention provides a compound of the invention as previously defined for use as a medicament.

In a further aspect the present invention provides the use of a compound of the invention in the preparation of a medicament for the treatment or prophylaxis of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms comprising administering a pharmacologically effective amount of a compound of the invention to a patient in need thereof.

The compounds of the present invention are particularly suitable for the treatment of obesity, e.g. by reduction of appetite and body weight, maintenance of weight reduction and prevention of rebound.

The compounds of the present invention may also be used to prevent or reverse medication-induced weight gain, e.g. weight gain caused by antipsychotic (neuroleptic) treatment(s). The compounds of the present invention may also be used to prevent or reverse weight gain associated with smoking cessation.

The compounds of the present invention may be suitable for use in treating the above indications in juvenile or adolescent patient populations.

The compounds of the present invention may also be suitable for use in the regulation of bone mass and bone loss and therefore useful in the treatment of osteoporosis and other bone diseases.

### Combination Therapy

The compounds of the invention may be combined with another therapeutic agent that is useful in the treatment of obesity such as other anti-obesity drugs, that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, or G-I motility.

The compounds of the invention may further be combined with another therapeutic agent that is useful in the treatment of disorders associated with obesity such as hypertension, hyperlipidaemias, dyslipidaemias, diabetes, sleep apnea, asthma, heart disorders, atherosclerosis, macro and micro vascular diseases, liver steatosis, cancer, joint disorders, and gallbladder disorders. For example, a compound of the present invention may be used in combination with a another therapeutic agent that lowers blood pressure or that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of the invention may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase is inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel.

According to an additional further aspect of the present invention there is provided a combination treatment comprising the administration of an effective amount of a compound of the invention, or a pharmaceutically acceptable salt thereof, optionally together with a pharmaceutically acceptable diluent or carrier, with the simultaneous, sequential or separate administration one or more of the following agents selected from:

a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-coagulant;
an omega-3 fatty acid;
another anti-obesity compound for example sibutramine, phentermine, orlistat, bupropion, ephedrine, thyroxine;
an antihypertensive compound for example an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic or a vasodilator;
a melanin concentrating hormone (MCH) modulator;
an NPY receptor modulator;
an orexin receptor modulator;
a phosphoinositide-dependent protein kinase (PDK) modulator; or
modulators of nuclear receptors for example LXR, FXR, RXR, GR, ERRα, β, PPARα, β, γ and RORalpha;
a monoamine transmission-modulating agent, for example a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA);
an antipsychotic agent for example olanzapine and clozapine;
a serotonin receptor modulator;
a leptin/leptin receptor modulator;
a ghrelin/ghrelin receptor modulator;
a DPP-IV inhibitor;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.
According to an additional further aspect of the present invention there is provided a combination treatment comprising the administration of an effective amount of a compound of the invention, or a pharmaceutically acceptable salt thereof, optionally together with a pharmaceutically acceptable diluent or carrier, with the simultaneous, sequential or separate administration of very low calorie diets (VLCD) or low-calorie diets (LCD).

Therefore in an additional feature of the invention, there is provided a method for the treatment of obesity and its associated complications in a warm-blooded animal, such as man, in need of such treatment which comprises administering to said animal an effective amount of a compound of the invention in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of the invention in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of the invention and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of the invention and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of the invention in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of the invention together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the invention and one of the other compounds described in this combination section, or a pharmaceutically acceptable salt, solvate, solvate of such a salt or a prodrug thereof, in the manufacture of a medicament for use in the treatment of obesity and its associated complications in a warm-blooded animal, such as man.

According to another feature of the invention there is provided the use of a compound of the invention and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the invention optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatohepatitis, osteoarthritis and some cancers) and psychiatric and neurological conditions.

It will be understood that there are medically accepted definitions of obesity and being overweight. A patient may be identified by, for example, measuring body mass index (BMI), which is calculated by dividing weight in kilograms by height in metres squared, and comparing the result with the definitions.

### Pharmacological Activity

Compounds of the present invention are active against the receptor product of the CB1 gene. The affinity of the compounds of the invention for central cannabinoid receptors is demonstrable in methods described in Devane et al, Molecular Pharmacology, 1988, 34, 605 or those described in WO01/70700 or EP 656354. Alternatively the assay may be performed as follows.

10 μg of membranes prepared from cells stably transfected with the CB1 gene were suspended in 200 μl of 100 mM NaCl, 5 mM MgCl₂, 1 mM EDTA, 50 mM HEPES (pH 7.4), 1 mM DTT, 0.1% BSA and 100 μM GDP. To this was added an EC80 concentration of agonist (CP55940), the required concentration of test compound and 0.1 μCi [³⁵S]-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 5 mM NaCl). Filters were then covered with scintillant and counted for the amount of [³⁵S]-GTPγS retained by the filter.

Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B−A)/1+((C/x)ÙD)) and the IC50 value determined as the concentration required to give half maximal inhibition of GTPγS binding under the conditions used.

The compounds of the present invention are active at the CB1 receptor (IC50<1 micromolar). Most preferred compounds have IC50<200 nanomolar.

The compounds of the invention are believed to be selective CB1 antagonists or inverse agonists. The potency, selectivity profile and side effect propensity may limit the clinical usefulness of hitherto known compounds with alleged CB1 antagonistic/inverse agonistic properties. In this regard, preclinical evaluation of compounds of the present invention in models of gastrointestinal and/or cardiovascular function indicates that they offer significant advantages compared to representative reference CB1 antagonist/inverse agonist agents.

The compounds of the present invention may provide additional benefits in terms of potency, selectivity profile, bioavailability, half-life in plasma, blood brain permeability, plasma protein binding (for example higher free fraction of drug) or solubility compared to representative reference CB1 antagonists/inverse agonist agents.

The utility of the compounds of the present invention in the treatment of obesity and related conditions is demonstrated by a decrease in body weight in cafeteria diet-induced obese mice. Female C57B1/6J mice were given ad libitum access to calorie-dense ‘cafeteria’ diet (soft chocolate/cocoa-type pastry, chocolate, fatty cheese and nougat) and standard lab chow for 8-10 weeks. Compounds to be tested were then administered systemically (iv, ip, sc or po) once daily for a minimum of 5 days, and the body weights of the mice monitored on a daily basis. Simultaneous assessment of adiposity was carried by means of DEXA imaging at baseline and termination of the study. Blood sampling was also carried out to assay changes in obesity-related plasma markers.

### EXAMPLES OF THE INVENTION

### Abbreviations

AcOH acetic acid
DCM dichloromethane
DMF dimethylformamide
DEA diethylamine

### DIEA N,N-diisopropylethylamine

DMAP 4-dimethylaminopyridine
EtOAc ethyl acetate
Et₃N triethylamine

### Ex or EX Example

LiHMDS lithium hexamethyldisilazide
MeOH methanol
MeCN acetonitrile
rt or RT room temperature
TEA triethylamine
THF tetrahydrofuran
t triplet
singlet
d doublet
q quartet
qvint quintet
m multiplet
br broad
bs broad singlet
dm doublet of multiplet
bt broad triplet
dd doublet of doublet

### General Experimental Procedures

Mass spectra were recorded on either a Micromass ZQ single quadrupole or a Micromass LCZ single quadrupole mass spectrometer both equipped with a pneumatically assisted electrospray interface (LC-MS). ¹H NMR measurements were performed on either a Varian Mercury 300 or a Varian Inova 500, operating at ¹H frequencies of 300 and 500 MHz respectively. Chemical shifts are given in ppm with CDCl₃ as internal standard. CDCl₃ is used as the solvent for NMR unless otherwise stated. Purification was performed on a semipreparative HPLC (High Performance Liquid Chromatography) with a mass triggered fraction collector, Shimadzu QP 8000 single quadrupole mass spectrometer equipped with 19×100 mm C8 column. The mobile phase used was, if nothing else is stated, acetonitrile and buffer (0.1 M ammonium acetate:acetonitrile 95:5).
For isolation of isomers, a Kromasil CN E9344 (250×20 mm i.d.) column was used. Heptane:ethyl acetate:DEA 95:5:0.1 was used as mobile phase (1 ml/min). Fraction collection was guided using a UV-detector (330 nm).
Typical HPLC-parameters for purity analysis:

### HPLC-system: Agilent 1100

### Column: Zorbax Eclipse XDB-C8 150×4.6 mm

Time of analysis: 15 min
Flow: 1.5 ml/min

### Mobilphase:

A: water, 5% MeOH
B: MeOH

### Temperature: 40° C.

### Detector: Uv 240 nm

### EXAMPLES

### General Procedure for Salt Preparation

The free base (usually 15-30 mg) was dissolved in butanone (usually 0.8-2 ml) and if necessary together with methanol (usually less than 1 ml). The solution was put in an ultrasonic bath (Decon FS200b). Acid (1 equiv. HCl or 1 equiv. methane sulphonic acid or 0.5 equiv. naphthalene-1,5-disulphonic acid, 1 equiv of sulphuric acid or 0.5 equiv. sulfuric acid), dissolved in methanol (usually 0.1-0.2 ml), was added. Heptane (usually 0.5-2 ml) was added dropwise and the resulting mixture was left in the ultrasonic bath. The solid formed was collected by filtration and was dried under high vacuum.

The melting points were determined on a Reichert melting point microscope and are uncorrected. It will be appreciated by those skilled in the art that the rate of heating can affect the melting point obtained and that certain salts, for example hydrochloride salts, may dissociate on slow heating so that ultimately the melting point obtained may be that of the free base or that of a mixture of free base and hydrochloride salt. In such cases combustion analysis may be used to confirm the identity of the salt. It will also be appreciated by those skilled in the art that the drying temperature should not be too high, for example greater than 45° C., otherwise decomposition of the salt may occur. Vacuum drying is preferred.

The following salts were formed as described in the general procedure by using the appropriate acid.

### Example 1

### 1-(4-Benzyloxy-phenyl)-2-(2,4-dichloro-phenyl)-5-methyl-1H-imidazole-4-carboxylic acid piperidin-1-ylamide

Hydrochloride: melting point 195-198° C.
Mesylate: melting point 196-198° C.
Hemi-1,5-naphthalenedisulphonate: melting point 219-225° C.

### Example 2

### Ethanesulfonic acid 4-[2-(2,4-dichloro-phenyl)-5-methyl-4-(piperidin-1-ylcarbamoyl)-imidazol-1-yl]-phenyl ester

Hydrochloride: melting point 94-101° C.
Mesylate: melting point 120-125° C.
Hemi-1,5-naphthalenedisulphonate: melting point 218-226° C.

### Example 3

### 4,4,4-Trifluoro-butane-1-sulfonic acid 4-[2-(2,4-dichloro-phenyl)-5-methyl-4-(piperidin-1-ylcarbamoyl)-imidazol-1-yl]-phenyl ester

Hydrochloride: melting point 113-118° C.
Mesylate: melting point 190-198° C.
Hemi-1,5-naphthalenedisulphonate: melting point 250-253° C.
Hydrogen sulphate: melting point 218-223° C.

### Example 4

### Pyridine-3-sulfonic acid 4-[2-(2,4-dichloro-phenyl)-5-methyl-4-(piperidin-1-ylcarbamoyl)-imidazol-1-yl]-phenyl ester

Hydrochloride: melting point: 205-209° C.
Hemi-1,5-naphthalenedisulphonate: melting point 167-170° C.

### Example 5

### 5-Chloro-thiophene-2-sulfonic acid 4-[2-(2,4-dichloro-phenyl)-5-methyl-4-(piperidin-1-ylcarbamoyl)-imidazol-1-yl]-phenyl ester

Hydrochloride: melting point: 112-118° C.
Mesylate: melting point 113-117° C.
Hemi-1,5-naphthalenedisulphonate: melting point 178-182° C.
Hydrogen sulphate: melting point 208-214° C.

### Example 6

### 3,3-Dimethyl-butane-1-sulfonic acid 4-[2-(2,4-dichloro-phenyl)-5-methyl-4-(piperidin-1-ylcarbamoyl)-imidazol-1-yl]-phenyl ester

Hydrochloride: melting point 152-156° C.
Mesylate: melting point 143-151° C.
Hemi-1,5-naphthalenedisulphonate: melting point 149-158° C.

### Example 7

### Thiophene-2-sulfonic acid 4-[2-(2,4-dichloro-phenyl)-5-methyl-4-(piperidin-1-ylcarbamoyl)-imidazol-1-yl]phenyl ester

Hydrochloride: melting point 107-112° C.
Hemi-1,5-naphthalenedisulphonate: melting point 172-180° C.
Hydrogen sulphate: melting point 188-193° C.

### Example 8

### 3,3,3-Trifluoro-propane-1-sulfonic acid 4-[2-(2,4-dichloro-phenyl)-5-methyl-4-(piperidin-1-ylcarbamoyl)-imidazol-1-yl]-phenyl ester

Hydrochloride: melting point 183-186° C.
Hydrogen sulphate: melting point 107-114° C.
Mesylate: melting point 190-194° C.
Hemi-1,5-naphthalenedisulphonate: melting point 253-256° C.
Ethylsulphonate: melting point 180-187° C.

### Example 9

### Propane-1-sulfonic acid 4-[2-(2,4-dichloro-phenyl)-5-methyl-4-(piperidin-1-ylcarbamoyl)-imidazol-1-yl]-phenyl ester

Hydrochloride: melting point 126-133° C.
Mesylate: melting point: 104-110° C.
Hemi-1,5-naphthalenedisulphonate: melting point 159-163° C.

### Preparation of Free Bases

### A

### Step 1.

### N-(4-Benzyloxyphenyl)-2,4-dichloro-benzamidine

4-Benzyloxyaniline hydrochloride (5.0 g, 21.2 mmol) was dropwise added to a solution of ethylmagnesium bromide (44.5 ml, 1M in THF, 44.5 mmol) in 25 ml dry THF under nitrogen atmosphere. After stirring for 20 minutes a solution of 2,4-dichlorobenzonitrile (3.65 g, 21.2 mmol) in 25 ml THF was added. The reaction mixture was stirred for 20 hours at room temperature. Water (50 ml) was carefully added. Extraction with EtOAc (2×100 ml), drying (Na₂SO₄), filtration and evaporation to dryness afforded 7.7 g (98%) of the title compound.

Step 2.

### 1-(4-Benzyloxyphenyl)-2-(2,4-dichlorophenyl)-5-methyl-1H-imidazole-4-carboxylic acid ethyl ester

To N-(4-benzyloxyphenyl)-2,4-dichlorobenzamidine, from Step 1 (6.88 g, 18.5 mmol) dissolved in 50 ml THF was added potassium carbonate (2.56 g, 18.5 mmol) and the suspension was stirred for 10 minutes. Ethyl-3-bromo-2-oxobutanoate (4.65 g, 22.2 mmol) was dropwise added over 1 hour, and the mixture was stirred for 66 hours at room temperature. The solution was filtered and evaporated to dryness. The residue was dissolved in acetic acid and refluxed for 1 hour. The mixture was cooled to room temperature, 100 ml water added and the product extracted with EtOAc (2×200 ml). The combined organic phases were washed with saturated sodium hydrogen carbonate, dried (Na₂SO₄), filtered and concentrated. Flash chromatography (silica, hexane:EtOAc 70:30, 60:40) afforded 5.75 g (65%) of the title compound as a pale yellow solid.

¹H NMR (CDCl₃): δ 7.5-7.2 (8H, m), 7.1-6.9 (4H, m), 5.1 (2H, s), 4.5 (2H, q), 2.5 (3H, s), 1.5 (3H, t), MS m/z 504 (M+Na), 985 (2 M+Na)

### Step 3.

### 1-(4-Benzyloxyphenyl)-2-(2,4-dichlorophenyl)-5-methyl-1H-imidazole-4-carboxylic acid

To a suspension of 1-(4-benzyloxyphenyl)-2-(2,4-dichlorophenyl)-5-methyl-1H-imidazole-4-carboxylic acid ethyl ester, from Step 2 (3.62 g, 7.5 mmol) in 60 ml methanol was added potassium hydroxide (4.05 g, 72 mmol) in water (20 ml), and the reaction mixture was refluxed for 2 hours. The mixture was cooled to room temperature, acidified to pH˜2 with 1 M HCl and extracted with ethyl acetate (2×200 ml). The combined organic phases were dried (Na₂SO₄), filtered and concentrated to give 3.38 g (99%) of the title compound.

### Step 4.

### 1-(4-Benzyloxyphenyl)-2-(2,4-dichlorophenyl)-5-methyl-1H-imidazole-4-carboxylic acid piperidin-1-ylamide

A solution of 1-(4-benzyloxyphenyl)-2-(2,4-dichlorophenyl)-5-methyl-1H-imidazole-4-carboxylic acid, from Step 3 (3.38 g, 7.5 mmol) in 60 ml CH₂Cl₂ was added 3 drops of DMF followed by oxalyl chloride (1.3 ml, 14.9 mmol). The mixture was refluxed for 2 hours, cooled to room temperature and evaporated to dryness. The residue was dissolved in 50 ml CH₂Cl₂ and cooled to 0° C. Triethylamine (2.1 ml, 14.9 mmol) was added followed by 1-aminopiperidine (0.9 ml, 8.2 mmol) and the mixture was stirred at room temperature for 2 hours. Water (300 ml) was added, the mixture extracted with CH₂Cl₂ (3×100 ml), dried (Na₂SO₄), filtered and concentrated. Flash chromatography (silica, hexane:EtOAc 1:2, EtOAc) afforded 2.94 g (74%) of the title compound as a white solid. ¹H NMR (CDCl₃): δ 7.5-7.2 (8H, m), 7.1-6.9 (4H, m), 5.1 (2H, s), 3.0-2.7 (4H, m), 2.5 (3H, s), 1.9-1.7 (4H, m), 1.6-1.4 (2H, m). MS m/z 558 (M+Na). HPLC: 96.5%.

### B

### Step 1.

### 2-(2,4-Dichlorophenyl)-1-(4-hydroxyphenyl)-5-methyl-1H-imidazole-4-carboxylic acid piperidin-1-ylamide

1-(4-Benzyloxyphenyl)-2-(2,4-dichlorophenyl)-5-methyl-1H-imidazole-4-carboxylic acid piperidin-1-ylamide, from A, Step 4 (2.78 g, 5.2 mmol) was dissolved in 80 ml CH₂Cl₂ and cooled to 0° C. Boron tribromide solution (1 M in CH₂Cl₂, 10.4 ml, 10.4 mmol) was added dropwise and the reaction mixture was stirred at room temperature for 1 hour. Water (200 ml) was added and the solution extracted with EtOAc (3×200 ml). The combined organic phases were dried (Na₂SO₄), filtered and concentrated. Flash chromatography (silica, hexane:EtOAc 1:3, EtOAc) afforded 1.34 g (58%) of the title compound as a white solid.

¹H NMR (CDCl₃): δ 8.6 (1H, bs), 7.4-7.1 (3H, m), 7.0-6.9 (4H, m), 3.0-2.8 (4H, m), 2.5 (3H, s), 1.8-1.6 (4H, m), 1.5-1.3 (2H, m).

Step 2

### Ethanesulfonic acid 4-[2-(2,4-dichlorophenyl)-5-methyl-4-(piperidin-1-ylcarbamoyl)-imidazol-1-yl]-phenyl ester

A solution of 2-(2,4-dichlorophenyl)-1-(4-hydroxyphenyl)-5-methyl-1H-imidazole-4-carboxylic acid piperidin-1-ylamide, from B, Step 1 (321 mg, 0.72 mmol) in 10 ml CH₂Cl₂ was cooled to 0° C. Triethylamine (101 μl, 0.72 mmol) was added followed by ethanesulfonyl chloride (69 μl, 0.72 mmol) and the reaction mixture was stirred at room temperature overnight. Water was added, the mixture extracted with CH₂Cl₂ (3×20 ml), dried (Na₂SO₄), filtered and concentrated. Flash chromatography (silica, hexane:EtOAc 1:3) afforded 230 mg (60%) of the title compound as a white solid.

¹H NMR (CDCl₃): δ 7.9 (1H, broad s), 7.4-7.1 (7H, m), 3.3 (2H, q), 3.0-2.8 (4H, m), 2.5 (3H, s), 1.9-1.7 (4H, m), 1.5 (3H, t), 1.5-1.4 (2H, m). MS m/z 560 (M+Na). HPLC: 97.0%.

### C

### Propane-1-sulfonic acid 4-[2-(2,4-dichlorophenyl)-5-methyl-4-(piperidin-1-ylcarbamoyl)-imidazol-1-yl]phenyl ester

A solution of 2-(2,4-dichlorophenyl)-1-(4-hydroxyphenyl)-5-methyl-1H-imidazole-4-carboxylic acid piperidin-1-ylamide, from B, Step 1 (320 mg, 0.72 mmol) in 10 ml CH₂Cl₂ was cooled to 0° C. Triethylamine (100 μl, 0.72 mmol) was added followed by 1-propanesulfonyl chloride (81 μl, 0.72 mmol) and the reaction mixture was stirred at room temperature overnight. Water was added, the mixture extracted with CH₂Cl₂ (3×20 ml), dried (Na₂SO₄), filtered and concentrated. Flash chromatography (silica, hexane:EtOAc 1:2) afforded 220 mg (56%) of the title compound as a white solid.

¹H NMR (CDCl₃): δ 7.9 (1H, broad s), 7.4-7.1 (7H, m), 3.3 (2H, m), 3.0-2.8 (4H, m), 2.5 (3H, s), 2.1-1.9 (2H, m), 1.9-1.7 (4H, m), 1.5-1.4 (2H, m), 1.2 (3H, t).

MS m/z 574 (M+Na). HPLC: 97.0%.

### D

### Butane-1-sulfonic acid 4-[2-(2,4-dichlorophenyl)-5-methyl-4-(piperidin-1-ylcarbamoyl)-imidazol-1-yl]phenyl ester

A solution of 2-(2,4-dichlorophenyl)-1-(4-hydroxyphenyl)-5-methyl-1H-imidazole-4-carboxylic acid piperidin-1-ylamide, B, Step 1 (320 mg, 0.72 mmol) in 10 ml CH₂Cl₂ was cooled to 0° C. Triethylamine (100 μl, 0.72 mmol) was added followed by 1-butanesulfonyl chloride (93 μl, 0.72 mmol) and the reaction mixture was stirred at room temperature overnight. Water was added and the mixture extracted with CH₂Cl₂ (3×20 ml), dried (Na₂SO₄), filtered and concentrated. Flash chromatography (silica, hexane:EtOAc 1:2) afforded 230 mg (57%) of the title compound as a white solid.

¹H NMR (CDCl₃): δ 7.9 (1H, bs), 7.4-7.1 (7H, m), 3.3 (2H, m), 3.0-2.8 (4H, m), 2.5 (3H, s), 2.1-1.9 (2H, m), 1.9-1.7 (4H, m), 1.6-1.4 (4H, m), 1.0 (3H, t)

MS m/z 588 (M+Na). HPLC: 96.0%.

### E

### 2-(2,4-Dichlorophenyl)-5-methyl-1-[4-(4,44-trifluorobutoxy)-phenyl]-1H-imidazole-4-carboxylic acid piperidin-1-ylamide

1-Iodo-4,4,4-trifluorobutane (376 mg, 1.58 mmol) was added dropwise to a suspension of 2-(2,4-dichlorophenyl)-1-(4-hydroxyphenyl)-5-methyl-1H-imidazole-4-carboxylic acid piperidin-1-ylamide, from B, Step 1 (351 mg, 0.79 mmol) and K₂CO₃ (218 mg, 1.58 mmol) in 50 ml acetone. The reaction mixture was refluxed overnight, cooled, filtered and concentrated. Flash chromatography (silica, hexane:EtOAc 1:2) afforded 200 mg (46%) of the title compound as a white solid.

¹H NMR (CDCl₃): δ 8.0 (1H, broad s), 7.4-7.2 (3H, m), 7.1-7.0 (2H, m), 6.9-6.8 (2H, m), 4.1-4.0 (2H, m), 3.0-2.9 (4H, m), 2.5-2.2 (5H, m), 2.2-2.0 (2H, m), 1.9-1.7 (4H, m), 1.6-1.4 (2H, m). MS m/z 578 (M+Na). HPLC: 99.4%.

### F

### 3,3,3-Trifluoropropane-1-sulfonic acid 4-[2-(2,4-dichlorophenyl)-5-methyl-4-(piperidin-1-ylcarbamoyl)imidazol-1-yl]phenyl ester

2-(2,4-Dichlorophenyl)-1-(4-hydroxyphenyl)-5-methyl-1H-imidazole-4-carboxylic piperidin-1-ylamide, from B, Step 1, (0.89 g, 2.00 mmol) was dissolved in dichloromethane (20 ml), cooled to 0° C. and triethylamine (0.35 ml, 2.4 mmol) added followed by 3,3,3-trifluoropropanesulfonyl chloride (prepared by an analogous method to that described in WO00/010968 for the butyl homologue) (0.35 ml, 2.40 mmol). The reaction mixture was stirred at room temperature overnight. TLC showed remaining starting material and so another portion of triethylamine and 3,3,3-trifluoropropanesulfonyl chloride was added and the reaction mixture stirred for additional 2 hrs. Water was added and the product was extracted with dichloromethane, dried (Na₂SO₄), filtered and concentrated. Flash chromatography (hexane:EtOAc 1:3-EtOAc) followed by recrystallisation (hexane:EtOAc) afforded 700 mg (59%) of the title compound as a colorless solid.

¹H NMR (CDCl₃): δ 7.40-7.10 (8H, m), 3.60-3.43 (2H, m), 3.02-2.70 (6H, m), 2.50 (3H, s), 1.92-1.70 (4H, m), 1.57-1.40 (2H, m). MS m/z 627 (M+Na). HPLC: 97.8%

### Example G

### 4,4,4-Trifluorobutane-1-sulfonic acid 4-[2-(2,4-dichlorophenyl)-5-methyl-4-(piperidin-1-ylcarbamoyl)imidazol-1-yl]-phenyl ester

2-(2,4-Dichlorophenyl)-1-(4-hydroxyphenyl)-5-methyl-1H-imidazole-4-carboxylic piperidin-1-ylamide, from B, Step 1 (0.49 g, 1.20 mmol) was dissolved in dichloromethane (20 ml), cooled to 0° C. and triethylamine (0.67 ml, 4.8 mmol) added followed by 4,4,4-trifluorobutane-1-sulfonyl chloride (prepared as described in WO00/010968) (0.38 g, 1.80 mmol). The reaction mixture was stirred at room temperature for 3 hrs. TLC showed remaining starting material and another portion of triethylamine and 4,4,4-trifluorobutane-1-sulfonyl chloride was added and the reaction mixture stirred overnight. Water was added, the product extracted with dichloromethane, dried (Na₂SO₄), filtered and concentrated. Flash chromatography (hexane:EtOAc 1:3-EtOAc) followed by recrystallisation (hexane:EtOAc) afforded 0.45 g (61%) of the title compound as a colorless solid.

¹H NMR (CDCl₃): δ 7.35-7.19 (8H, m), 3.40 (2H, m), 3.05-2.90 (4H, m), 2.78-2.20 (7H, s and m), 1.92-1.70 (4H, m), 1.57-1.40 (2H, m). MS m/z 641 (M+Na). HPLC: 98.6%

### H

### 4-[2-(2,4-dichlorophenyl)-5-methyl-4-[(piperidin-1-ylamino)carbonyl]-1H-imidazol-1-yl]phenyl thiophene-2-sulfonate

2-(2,4-Dichlorophenyl)-1-(4-hydroxyphenyl)-5-methyl-N-piperidin-1-yl-1H-imidazole-4-carboxamide, prepared as described in B, Step 1 (100 mg, 0.22 mmol) and triethylamine (0.31 ml, 2.25 mmol) in dichloromethane (2.5 ml) were cooled to −78° C. 2-Thiophenesulfonyl chloride (287 mg, 1.57 mmol) dissolved in dichloromethane (2.5 ml) was carefully added to the reaction mixture. The resulting mixture was stirred at −78° C. for 1 h, and at room temperature overnight. Water was added to the reaction, the phases were separated and the organic phase washed with water and dried. The solvent was removed under reduced pressure and separation by preparatory HPLC gave the title compound (110 mg, 83%) as a solid.

¹H NMR (400 MHz) δ 7.89 (s, NH), 7.75-7.74 (m, 1H), 7.55-7.54 (m, 1H), 7.35-7.34 (m, 1H), 7.30-7.25 (m, 2H), 7.13-7.11 (m, 1H), 7.07 (d, 4H), 2.90-2.86 (m, 4H), 1.80-1.75 (m, 4H), 1.48-1.42 (m, 2H). MS m/z 591 (M+H)⁺.

### I

### 4-{2-(2,4-dichlorophenyl)-5-methyl-4-[(piperidin-1-ylamino)carbonyl]-1H-imidazol-1-yl}phenyl pyridine-3-sulfonate

2-(2,4-Dichlorophenyl)-1-(4-hydroxyphenyl)-5-methyl-N-piperidin-1-yl-1H-imidazole-4-carboxamide, prepared as described in B, Step 1 (100 mg, 0.22 mmol) and triethylamine (0.31 ml, 2.25 mmol) in dichloromethane (5.0 ml) were cooled to −78° C. 3-Pyridinesulfonyl chloride (144 mg, 0.67 mmol) was added in small portions to the reaction mixture. The resulting mixture was stirred at −78° C. for 1 h, and at room temperature overnight. Water was added to the reaction, the phases were separated and the organic phase washed with water and dried. The solvent was removed under reduced pressure and separation by preparatory HPLC gave the title compound (110 mg, 84%) as a solid.

¹H NMR (400 MHz) δ 8.96 (s, 1H), 8.92 (s, 1H), 8.09-8.06 (m, 1H), 7.89 (s, 1H), 7.51-7.49 (m, 1H), 7.36 (d, 1H), 7.30-7.25 (m, 2H), 7.06 (s, 4H), 2.88-2.84 (m, 4H), 2.48 (s, 3H), 1.79-1.74 (m, 4H), 1.47-1.41 (m, 2H). MS m/z 586 (+H)⁺.

### J

### 4-{2-(2,4-dichlorophenyl)-5-methyl-4-[(piperidin-1-ylamino)carbonyl]-1H-imidazol-1-yl}phenyl 5-chlorothiophene-2-sulfonate

2-(2,4-Dichlorophenyl)-1-(4-hydroxyphenyl)-5-methyl-N-piperidin-1-yl-1H-imidazole-4-carboxamide, prepared as described in B, Step 1 (100 mg, 0.22 mmol) and triethylamine (0.16 ml, 1.12 mmol) in dichloromethane (2.5 ml) were cooled to −78° C. 5-Chlorothiophen-2-sulfonyl chloride (244 mg, 1.12 mmol) in dichloromethane (2.5 ml) was carefully added to the reaction mixture. The resulting mixture was stirred at −78° C. for 1 h, and at room temperature overnight. Water was added to the reaction, the phases were separated and the organic phase washed with water and dried. The solvent was removed under reduced pressure and separation by preparatory HPLC gave the title compound (84 mg, 60%) as a solid.

¹H NMR (400 MHz) δ 7.90 (s, NH), 7.34-7.26 (m, 4H), 7.10 (d, 4H), 6.96 (d, 1H), 2.88-2.85 (m, 4H), 2.49 (s, 3H), 1.79-1.74 (m, 4H), 1.47-1.42 (m, 2H). MS m/z 625 (M+H)⁺.

### K

### 4-{2-(2,4-dichlorophenyl)-5-methyl-4-[(piperidin-1-ylamino)carbonyl]-1H-imidazol-1-yl}phenyl 3-methylbutane-1-sulfonate

2-(2,4-Dichlorophenyl)-1-(4-hydroxyphenyl)-5-methyl-N-piperidin-1-yl-1H-imidazole-4-carboxamide, prepared as described in B, Step 1 (50 mg, 0.11 mmol) was dissolved in dichloromethane (3.0 ml), cooled to 0° C. and triethylamine (20 μl, 0.13 mmol) was added to the mixture. The resulting mixture was cooled to −78° C. and 3-methylbutane-1-sulfonyl chloride (23 mg, 0.13 mmol) was carefully added. The reaction was stirred at −78° C. for 1.5 h. Water was added to the reaction, the product extracted with dichloromethane and dried. The solvent was removed under reduced pressure and separation by preparatory HPLC gave the title compound (46 mg, 71%) as a solid.

¹H NMR (400 MHz) δ 7.86 (s, NH), 7.30-7.20 (m, 5H), 7.12-7.09 (m, 2H), 3.26-3.22 (m, 2H), 2.86-2.80 (m, 4H), 2.46 (s, 3H), 1.86-1.80 (m, 3H), 1.77-1.69 (m, 4H), 1.45-1.37 (m, 2H), 0.93 (d, 6H). MS m/z 579 (M+H)⁺.

### L

### 4-{2-(2,4-dichlorophenyl)-5-methyl-4-[(piperidin-1-ylamino)carbonyl]-1H-imidazol-1-yl}phenyl 3,3-dimethylbutane-1-sulfonate

2-(2,4-Dichlorophenyl)-1-(4-hydroxyphenyl)-5-methyl-N-piperidin-1-yl-1H-imidazole-4-carboxamide, prepared as described in B, Step 1 (50 mg, 0.11 mmol) was dissolved in dichloromethane (3.0 ml), cooled to 0° C. and triethylamine (20 μl, 0.13 mmol) was added to the mixture. The resulting mixture was cooled to −78° C. and 3,3-dimethylbutane-1-sulfonyl chloride (25 mg, 0.13 mmol) was carefully added. The reaction was stirred at −78° C. for 2 h. Water was added to the reaction, the product extracted with dichloromethane and dried. The solvent was removed under reduced pressure and separation by preparatory HPLC gave the title compound (46 mg, 69%) as a solid.

¹H NMR (400 MHz) δ 7.85 (s, NH), 7.30-7.20 (m, 5H), 7.13-7.09 (m, 2H), 3.24-3.18 (m, 2H), 2.86-2.81 (m, 4H), 2.46 (s, 3H), 1.86-1.80 (m, 2H), 1.76-1.69 (m, 5H), 1.44-1.37 (m, 2H), 0.92 (s, 9H). MS m/z 593 (M+H)⁺.

### M

### Step 1: 1-[4-(Benzyloxy)phenyl]-2-(2,4-dichlorophenyl)-5-methyl-N-[5-(trifluoromethyl)pyridin-2-yl]-1H-imidazole-4-carboxamide

2-Amino-5-(trifluoromethyl)pyridine (404 mg, 2.49 mmol) was dissolved in dichloromethane (2.5 ml) under argon and trimethylaluminum (1.25 ml, 2.0 M in toluene, 2.5 mmol) was carefully added during 5 min. The solution was stirred at ambient temperature for 1.5 h and, as a result, a 0.66 M solution of an amidation reagent was obtained. 3.75 ml (2.5 mmol) of this stock solution was added to ethyl 1-[4-(benzyloxy)phenyl]-2-(2,4-dichlorophenyl)-5-methyl-1H-imidazole-4-carboxylate, prepared as described in A, Step 2, (400 mg, 0.83 mmol) and the reaction solution was stirred at 45° C. overnight. The reaction solution was cooled to 0° C. and quenched with HCl (aq, 2 M, 7.5 ml). The mixture was diluted with dichloromethane and neutralized by addition of KOH (aq, 2 M). The organic phase was separated and the aqueous phase was extracted further with dichloromethane. The collected organic phases were washed with H₂O before drying with Na₂SO₄. The solvent was removed under reduced pressure and purification by preparatory HPLC gave the title compound (319 mg, 64%) as a solid.

¹H NMR (400 MHz) δ 9.89 (s, NH), 8.54 (s, 1H), 8.50 (d, 1H), 7.92-7.88 (m, 1H), 7.40-7.33 (m, 5H), 7.27-7.20 (m, 3H), 7.03-6.91 (m, 4H), 5.02 (s, 2H), 2.50 (s, 3H).

MS m/z 597 (M+H)⁺.

### Step 2: 2-(2,4-dichlorophenyl)-1-(4-hydroxyphenyl)-5-methyl-N-[5-(trifluoromethyl)pyridin-2-yl]-1H-imidazole-4-carboxamide

1-[4-(benzyloxy)phenyl]-2-(2,4-dichlorophenyl)-5-methyl-N-[5-(trifluoromethyl)pyridin-2-yl]-1H-imidazole-4-carboxamide (319 mg, 0.53 mmol) was dissolved in hydrogen bromide (7.5 ml, 4.1 M in acetic acid, 30.75 mmol) and the reaction mixture was stirred at room temperature for 4 h. The acetic acid was co-evaporated with ethanol, the residue neutralized with ammonia and dissolved in methanol. Purification by flash chromatography gave the title compound (266 mg, 98%).

¹H NMR (400 MHz) δ 10.36 (s, NH el. OH), 10.09 (s, NH el. OH), 8.89 (s, 1H), 8.69 (d, 1H), 8.48-8.43 (m, 1H), 7.87-7.80 (m, 2H), 7.67 (d, 1H), 7.41 (d, 2H), 7.06 (d, 2H), 2.65 (s, 3H). MS m/z 507 (M+H)⁺.

### Step 3: 4-[2-(2,4-dichlorophenyl)-5-methyl-4-({[5-(trifluoromethyl)pyridin-2-yl]amino}carbonyl)-1H-imidazol-1-yl]phenyl 3,3,3-trifluoropropane-1-sulfonate

2-(2,4-dichlorophenyl)-1-(4-hydroxyphenyl)-5-methyl-N-[5-(trifluoromethyl)pyridin-2-yl]-1H-imidazole-4-carboxamide (136 mg, 0.27 mmol) and triethylamine (40 μl, 0.32 mmol) in dichloromethane (4.0 ml) were cooled to −78° C. 3,3,3-trifluoropropane-1-sulfonyl chloride (63 mg, 0.32 mmol) was carefully added to the reaction mixture. The resulting mixture was stirred at −78° C. for 1 h, and then allowed to reach room temperature. Water was added to the reaction, and the phases were separated. The organic phase was washed with NaHCO₃, brine and dried with Na₂SO₄. The solvent was removed under reduced pressure and separation by preparatory HPLC gave the title compound (88 mg, 49%) as a solid.

¹H NMR (400 MHz) δ 9.87 (s, NH), 8.55 (s, 1H), 8.49 (d, 1H), 7.93-7.89 (m, 1H), 7.34-7.18 (m, 7H), 3.53 (m, 2H), 2.85-2.73 (m, 2H), 2.54 (s, 3H). MS m/z 667 (M+H)⁺.

### N

### 4-[2-(2,4-dichlorophenyl)-5-methyl-4-({[5-(trifluoromethyl)pyridin-2-yl]amino}carbonyl)-1H-imidazol-1-yl]phenyl 3-methylbutane-1-sulfonate

2-(2,4-dichlorophenyl)-1-(4-hydroxyphenyl)-5-methyl-N-[5-(trifluoromethyl)pyridin-2-yl]-1H-imidazole-4-carboxamide, prepared as described in M, Step 3 (139 mg, 0.27 mmol) and triethylamine (46 μl, 0.33 mmol) in dichloromethane (4.0 ml) were cooled to −78° C. 3-Methylbutane-1-sulfonyl chloride (56 mg, 0.33 mmol) was carefully added to the reaction mixture. The resulting mixture was stirred at −78° C. for 1 h, and then allowed to reach room temperature. Water was added to the reaction, and the phases were separated. The organic phase was washed with NaHCO₃, brine and dried with Na₂SO₄. The solvent was removed under reduced pressure and separation by preparatory HPLC gave the title compound (81 mg, 46%) as a solid.

¹H NMR (400 MHz) δ 9.87 (s, NH), 8.55 (s, 1H), 8.49 (d, 1H), 7.93-7.89 (m, 1H), 7.34-7.14 (m, 7H), 3.29-3.24 (m, 2H), 2.53 (s, 3H), 1.88-1.81 (m, 2H), 1.79-1.69 (m, 1H), 0.95 (d, 6H). MS m/z 641 (M+H)⁺.

1. A compound selected from:
1-(4-benzyloxyphenyl)-2-(2,4-dichlorophenyl)-5-methyl-1H-imidazole-4-carboxylic acid piperidin-1-ylamide;
ethanesulfonic acid 4-[2-(2,4-dichlorophenyl)-5-methyl-4-(piperidin-1-ylcarbamoyl)-imidazol-1-yl]phenyl ester;
propane-1-sulfonic acid 4-[2-(2,4-dichlorophenyl)-5-methyl-4-(piperidin-1-ylcarbamoyl)-imidazol-1-yl]phenyl ester;
butane-1-sulfonic acid 4-[2-(2,4-dichlorophenyl)-5-methyl-4-(piperidin-1-ylcarbamoyl)-imidazol-1-yl]phenyl ester;
2-(2,4-dichlorophenyl)-5-methyl-1-[4-(4,4,4-trifluorobutoxy)phenyl]-1H-imidazole-4-carboxylic acid piperidin-1-ylamide;
3,3,3-trifluoropropane-1-sulfonic acid 4-[2-(2,4-dichlorophenyl)-5-methyl-4-(piperidin-1-ylcarbamoyl)imidazol-1-yl]phenyl ester;
4,4,4-trifluorobutane-1-sulfonic acid 4-[2-(2,4-dichlorophenyl)-5-methyl-4-(piperidin-1-ylcarbamoyl)imidazol-1-yl]phenyl ester;
4-{2-(2,4-dichlorophenyl)-5-methyl-4-[(piperidin-1-ylamino)carbonyl]-1H-imidazol-1-yl}phenyl thiophene-2-sulfonate;
4-{2-(2,4-dichlorophenyl)-5-methyl-4-[(piperidin-1-ylamino)carbonyl]-1H-imidazol-1-yl}phenyl pyridine-3-sulfonate;
4-{2-(2,4-dichlorophenyl)-5-methyl-4-[(piperidin-1-ylamino)carbonyl]-1H-imidazol-1-yl}phenyl pyridine-3-sulfonate;
4-{2-(2,4-dichlorophenyl)-5-methyl-4-[(piperidin-1-ylamino)carbonyl]-1H-imidazol-1-yl}phenyl 3-methylbutane-1-sulfonate;
4-{2-(2,4-dichlorophenyl)-5-methyl-4-[(piperidin-1-ylamino)carbonyl]-1H-imidazol-1-yl}phenyl 3,3-dimethylbutane-1-sulfonate;
4-[2-(2,4-dichlorophenyl)-5-methyl-4-({[5-(trifluoromethyl)pyridin-2-yl]amino}carbonyl)-1H-imidazol-1-yl]phenyl 3,3,3-trifluoropropane-1-sulfonate; and
4-[2-(2,4-dichlorophenyl)-5-methyl-4-({[5-(trifluoromethyl)pyridin-2-yl]amino}carbonyl)-1H-imidazol-1-yl]phenyl 3-methylbutane-1-sulfonate;
in the form of a methanesulphonate salt (mesylate salt), a hemi-1,5-naphthalenedisulphonate salt, a hemi-1,2-ethanedisulfonic acid salt, a hydrochloride salt, an ethylsulphonate salt, a nitrate salt, a sulphate salt or a hydrogen sulphate salt.

2. A compound selected from:
1-(4-benzyloxyphenyl)-2-(2,4-dichlorophenyl)-5-methyl-1H-imidazole-4-carboxylic acid piperidin-1-ylamide hydrochloride;
1-(4-benzyloxyphenyl)-2-(2,4-dichlorophenyl)-5-methyl-1H-imidazole-4-carboxylic acid piperidin-1-ylamide mesylate;
1-(4-benzyloxyphenyl)-2-(2,4-dichlorophenyl)-5-methyl-1H-imidazole-4-carboxylic acid piperidin-1-ylamide hemi-1,5-naphthalenedisulphonate;
ethanesulfonic acid 4-[2-(2,4-dichlorophenyl)-5-methyl-4-(piperidin-1-ylcarbamoyl)-imidazol-1-yl]phenyl ester hydrochloride;
ethanesulfonic acid 4-[2-(2,4-dichlorophenyl)-5-methyl-4-(piperidin-1-ylcarbamoyl)-imidazol-1-yl]phenyl ester mesylate;
ethanesulfonic acid 4-[2-(2,4-dichlorophenyl)-5-methyl-4-(piperidin-1-ylcarbamoyl)-imidazol-1-yl]phenyl ester hemi-1,5-naphthalenedisulphonate;
4,4,4-trifluorobutane-1-sulfonic acid 4-[2-(2,4-dichlorophenyl)-5-methyl-4-(piperidin-1-ylcarbamoyl)imidazol-1-yl]phenyl ester hydrochloride;
4,4,4-trifluorobutane-1-sulfonic acid 4-[2-(2,4-dichlorophenyl)-5-methyl-4-(piperidin-1-ylcarbamoyl)imidazol-1-yl]phenyl ester 4,4,4-trifluorobutane-1-sulfonic acid mesylate;
4-[2-(2,4-dichlorophenyl)-5-methyl-4-(piperidin-1-ylcarbamoyl)imidazol-1-yl]phenyl ester hemi-1,5-naphthalenedisulphonate;
4,4,4-trifluorobutane-1-sulfonic acid 4-[2-(2,4-dichlorophenyl)-5-methyl-4-(piperidin-1-ylcarbamoyl)imidazol-1-yl]phenyl ester hydrogen sulphate;
pyridine-3-sulfonic acid 4-[2-(2,4-dichlorophenyl)-5-methyl-4-(piperidin-1-ylcarbamoyl)-imidazol-1-yl]phenyl ester hydrochloride;
pyridine-3-sulfonic acid 4-[2-(2,4-dichlorophenyl)-5-methyl-4-(piperidin-1-ylcarbamoyl)-imidazol-1-yl]phenyl ester hemi-1,5-naphthalenedisulphonate;
5-chlorothiophene-2-sulfonic acid 4-[2-(2,4-dichlorophenyl)-5-methyl-4-(piperidin-1-ylcarbamoyl)imidazol-1-yl]phenyl ester hydrochloride;
5-chlorothiophene-2-sulfonic acid 4-[2-(2,4-dichlorophenyl)-5-methyl-4-(piperidin-1-ylcarbamoyl)imidazol-1-yl]phenyl ester mesylate;
5-chlorothiophene-2-sulfonic acid 4-[2-(2,4-dichlorophenyl)-5-methyl-4-(piperidin-1-ylcarbamoyl)imidazol-1-yl]phenyl ester hemi-1,5-naphthalenedisulphonate;
5-chlorothiophene-2-sulfonic acid 4-[2-(2,4-dichlorophenyl)-5-methyl-4-(piperidin-1-ylcarbamoyl)imidazol-1-yl]phenyl ester hydrogen sulphate;
3,3-dimethylbutane-1-sulfonic acid 4-[2-(2,4-dichlorophenyl)-5-methyl-4-(piperidin-1-ylcarbamoyl)imidazol-1-yl]phenyl ester hydrochloride;
3,3-dimethylbutane-1-sulfonic acid 4-[2-(2,4-dichlorophenyl)-5-methyl-4-(piperidin-1-ylcarbamoyl)imidazol-1-yl]phenyl ester mesylates;
3,3-dimethylbutane-1-sulfonic acid 4-[2-(2,4-dichlorophenyl)-5-methyl-4-(piperidin-1-ylcarbamoyl)imidazol-1-yl]phenyl ester hemi-1,5-naphthalenedisulphonate;
thiophene-2-sulfonic acid 4-[2-(2,4-dichlorophenyl)-5-methyl-4-(piperidin-1-ylcarbamoyl) imidazol-1-yl]phenyl ester hydrochloride;
thiophene-2-sulfonic acid 4-[2-(2,4-dichlorophenyl)-5-methyl-4-(piperidin-1-ylcarbamoyl) imidazol-1-yl]phenyl ester hemi-1,5-naphthalenedisulphonate;
thiophene-2-sulfonic acid 4-[2-(2,4-dichlorophenyl)-5-methyl-4-(piperidin-1-ylcarbamoyl) imidazol-1-yl]phenyl ester hydrogen sulphate;
3,3,3-trifluoropropane-1-sulfonic acid 4-[2-(2,4-dichlorophenyl)-5-methyl-4-(piperidin-1-ylcarbamoyl)imidazol-1-yl]phenyl ester hydrochloride;
3,3,3-trifluoropropane-1-sulfonic acid 4-[2-(2,4-dichlorophenyl)-5-methyl-4-(piperidin-1-ylcarbamoyl)imidazol-1-yl]phenyl ester hydrogen sulphate;
3,3,3-trifluoropropane-1-sulfonic acid 4-[2-(2,4-dichlorophenyl)-5-methyl-4-(piperidin-1-ylcarbamoyl)imidazol-1-yl]phenyl ester mesylate;
3,3,3-trifluoropropane-1-sulfonic acid 4-[2-(2,4-dichlorophenyl)-5-methyl-4-(piperidin-1-ylcarbamoyl)imidazol-1-yl]phenyl ester hemi-1,5-naphthalenedisulphonate;
3,3,3-trifluoropropane-1-sulfonic acid 4-[2-(2,4-dichlorophenyl)-5-methyl-4-(piperidin-1-ylcarbamoyl)imidazol-1-yl]phenyl ester ethylsulphonate;
propane-1-sulfonic acid 4-[2-(2,4-dichlorophenyl)-5-methyl-4-(piperidin-1-ylcarbamoyl)imidazol-1-yl]phenyl ester hydrochloride;
propane-1-sulfonic acid 4-[2-(2,4-dichlorophenyl)-5-methyl-4-(piperidin-1-ylcarbamoyl)imidazol-1-yl]phenyl ester mesylates; and
propane-1-sulfonic acid 4-[2-(2,4-dichlorophenyl)-5-methyl-4-(piperidin-1-ylcarbamoyl)imidazol-1-yl]phenyl ester hemi-1,5-naphthalenedisulphonate.

3. A compound of formula I 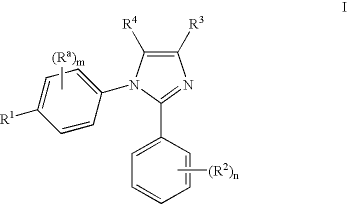
in which
R¹ represents
a) a C₁₋₁₀alkoxy group optionally substituted by one or more fluoro
b) a group of formula phenyl(CH₂)ₚO— in which p is 1, 2 or 3 and the phenyl ring is optionally substituted by 1, 2 or 3 groups represented by Z,
c) a group R⁵S(O)₂O or R⁵S(O)₂NH in which R⁵ represents a C₁₋₁₀alkyl group optionally substituted by one or more fluoro, or R⁵ represents phenyl or a heteroaryl group each of which is optionally substituted by 1, 2 or 3 groups represented by Z or
d) a group of formula (R⁶)₃ Si in which R⁶ represents a C₁₋₆alkyl group which may be the same or different;
R^{a} represents halo, a C₁₋₃alkyl group or a C₁₋₃alkoxy group;
m is 0, 1, 2 or 3;
R² represents a C₁₋₃alkyl group, a C₁₋₃alkoxy group, hydroxy, nitro, cyano or halo
n is 0, 1, 2 or 3;
R³ represents
a) a group X—Y—NR⁷R⁸ in which
X is CO or SO₂,
Y is absent or represents NH optionally substituted by a C₁₋₃alkyl group; and
R⁷ and R⁸ independently represent:
a C₁₋₆alkyl group optionally substituted by 1, 2, or 3 groups represented by W;
a C₃₋₁₅cycloalkyl group optionally substituted by 1, 2, or 3 groups represented by W;
an optionally substituted (C₃₋₁₅cycloalkyl)C₁₋₃alkylene group optionally substituted by 1, 2, or 3 groups represented by W;
a group —(CH₂)ᵣ(phenyl), in which r is 0, 1, 2, 3 or 4, s is 1 when r is 0 otherwise s is 1 or 2 and the phenyl groups are optionally independently substituted by one, two or three groups represented by Z;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;
a group —(CH₂), Het in which t is 0, 1, 2, 3 or 4, and the alkylene chain is optionally substituted by one or more C₁₋₃alkyl groups and Het represents a heteroaryl group optionally substituted by one, two or three groups selected from a C₁₋₅alkyl group, a C₁₋₅alkoxy group or halo wherein the alkyl and alkoxy group are optionally independently substituted by one of more fluoro;
or R⁷ represents H and R⁸ is as defined above;
or R⁷ and R⁸ together with the nitrogen atom to which they are attached represent a saturated or partially unsaturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy, fluoro or benzyl; or
b) oxazolyl, isoxazolyl, thiazolyl, isothiazolyl, oxadiazolyl, thiadiazolyl, pyrrolyl, pyrazolyl, imidazolyl, triazolyl, tetrazolyl, thienyl, furyl or oxazolinyl, each optionally substituted by 1, 2 or 3 groups Z;
R⁴ represents H, a C₁₋₆alkyl group, a C₁₋₆alkoxy group or a C₁₋₆alkoxyC₁₋₆alkylene group which contains a maximum of 6 carbon atoms, each of which groups is optionally substituted by one or more fluoro or cyano;
Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, C₁₋₃alkylsulphonyl, C₁₋₃alkoxycarbonyl, carboxy, cyano, carbamoyl, mono or di C₁₋₃alkyl carbamoyl and acetyl; and
W represents hydroxy, fluoro, a C₁₋₃alkyl group, a C₁₋₃alkoxy group, amino, mono or di C₁₋₃alkylamino, or a heterocyclic amine selected from morpholinyl, pyrrolidinyl, piperidinyl or piperazinyl in which the heterocyclic amine is optionally substituted by a C₁₋₃alkyl group or hydroxyl;
with the proviso that when n is 1 then R² is not methoxy in either the 2-position or the 4-position of the phenyl ring and the further proviso that R¹ is not methylsulfonylamino, methoxy or CF₃O—; in the form of a methanesulphonate salt (mesylate salt), a hemi-1,5-naphthalenedisulphonate salt, a hemi-1,2-ethanedisulfonic acid salt, an ethylsulphonate salt, or a nitrate salt.

4. (canceled)

5. A pharmaceutical formulation comprising a compound as claimed in any one of claims 1 to 3 and a pharmaceutically acceptable adjuvant, diluent or carrier.

6. (canceled)

7. A method of treating obesity, psychiatric disorders, psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy and related conditions, neurological disorders, Parkinson's Disease, Huntington's Chorea and Alzheimer's Disease, immune, cardiovascular, reproductive and endocrine disorders, septic shock, diseases related to the respiratory and gastrointestinal system, and extended abuse, addiction and/or relapse indications, comprising administering a pharmacologically effective amount of a compound as claimed in any one of claims 1 to 3 to a patient in need thereof.

8. (canceled)
